(19) United States
(12) Patent Application Publication
(10) Pub. No.: US 2026/0174546 A1
(43) Pub. Date: Jun. 25, 2026

(54) POSTERIOR CHAMBER PHAKIC INTRAOCULAR LENS (IOL) AND MANUFACTURING METHOD OF THE SAME

(71) Applicant: PHYSIOL, ANGLEUR (BE)

(72) Inventors: Christophe PAGNOULLE, Verviers (BE); Suad REDZOVIC, Jupille sur Meuse (BE); Renzo BUCCA PUY, Liège (BE); Mikhail BOUKHNY, Laguna Niguel, CA (US)

(73) Assignee: PHYSIOL, ANGLEUR (BE)

(21) Appl. No.: 19/012,591

(22) Filed: Jan. 7, 2025

(30) Foreign Application Priority Data
Dec. 24, 2024 (BE) 2024/5938

(51) Int. Cl. A61F 2/16

(52) U.S. Cl. CPC A61F 2/16; A61F 2002/1689; A61F 2240/002

(57) ABSTRACT

A posterior chamber phakic intraocular lens (IOL) having an inherent vault height independent from its external diameter, and a manufacturing method of such an IOL, are described.

### TECHNICAL FIELD

This application relates to an intraocular lens (noted “IOL” throughout the present document). More specifically, this application concerns a posterior chamber phakic IOL and corresponding manufacturing method.

### BACKGROUND

Generally speaking, phakic IOLs are IOLs intended to be placed in an eye in order to correct defects of vision. These are generally implanted in eyes of young patients, as a complement to the natural crystalline lens. Phakic IOLs that are intended to be implanted in an area of the eye between a posterior surface of the iris and an anterior surface of the crystalline lens are called posterior chamber phakic IOLs. These are supported around the ciliary body of the eye.

A limit in implantation of such an IOL lies in the fact that it is likely to be positioned differently from one eye to the other on the basis of parameters, especially the anatomy of the posterior chamber, the size of which usually varies by several millimeters from one patient to another. In particular, implantation of a posterior chamber phakic IOL, the size of which may not be adapted, would risk leading to more of less serious medical complications for the patient, such as:
for example, in a case where the phakic IOL is too small with respect to the size of an available posterior chamber anatomical space for IOL implantation: a contact of the IOL with the crystalline lens that generates a cataract of the eye, or a loss in the corrective power of the phakic IOL, or that has an impact on vision precision;
or for example, in a case where the phakic IOL is too large with respect to the size of this anatomical space: a pupillary block, a glaucoma, an inflammation, an iris depigmentation, or a depression between the anterior and posterior chambers of the eye after a pupillary block.

In order to overcome this problem known as “sizing issue”, it is known to produce various sizes of phakic IOLs based on general eyes anatomy. Each size corresponds a proportional dimensioning of the phakic IOL corresponding to one choice of its external diameter. The positioning of the IOL along the optical axis in the patient's eye (defining its positioning with respect to the eye's iris and crystalline lens) is targeted based on an expected radial compression on the IOL into the eye, as it is represented on FIG. 6 hereafter introduced. This solution, however, suffers from shortcomings in terms of the stability of the position of such an IOL once it is implanted in an eye due to uncertainties on radial compressions exerted on IOL haptics. Furthermore, anatomical measuring errors, irregularities of the ciliary body and/or sulcus and IOL misplacements during the implantation are also likely to lead to bad IOL positioning in the eye's posterior chamber, so that improvements are still needed regarding this “sizing issue”.

As an attempt to solve this problem, document WO 2020/035534 A1 discloses a single sized posterior chamber phakic IOL comprising a double haptic structure made of a peripheral haptic part comprising support elements arranged to lie on the ciliary zonule and elongated thin flexible haptics, each with a proximal extremity mounted on a proximal portion of the peripheral haptic part and with a free distal extremity to hook the IOL into the eye's ciliary sulcus. The haptics are deemed to allow for compensating any (or almost) size variation of the posterior chamber anatomical space, so that to stabilize the IOL's position in the eye. The haptics as being thin, flexible and elongated are nevertheless quite difficult to see and to maneuver during the implantation process in the eye, so that they are also likely to lead to IOL misplacements.

The document WO 2023/021225 A2 then further disclosed a posterior chamber phakic IOL easier to implant in an eye and still adapted to a wide class of eye's anatomy, without necessarily being adapted to all eye's anatomy and all posterior chamber size variations. This IOL comprises a double haptic structure, but this time elongated flexible haptics have their two extremities (considered as from an extension curve) mounted on a distal support element of the peripheral haptic part, so that defining so called footplates. The IOL can be stabilized in the eye's ciliary body by a distal lateral border of each footplate. Although mounting said extremities on the distal support element reduces the footplates adaptability in comparison with long, thin and freely ended flexible haptics as in the document WO 2020/035534 A1, this new geometry makes the footplates easier to see and to maneuver during the IOL implantation process. A small amount of IOL sizes 1 -to-1 associated to the same amount of external diameters (for instance three external diameters of 12.7, 13.2 and 13.6 mm) for such a posterior chamber phakic IOL are disclosed as being sufficient to cover possible eyes anatomies.

Although the disclosure of document WO 2023/021225 A2 is a major achievement in the technical field, improvements may still be needed with respect to the “sizing issue” as the phakic IOL positioning into the posterior chamber may not precisely be determined only on basis of the IOL diameter sizing and corresponding expected radial compressions exerted on the IOL haptic structures as mentioned above.

### BRIEF SUMMARY

An object of the invention is to provide a posterior chamber phakic IOL with a more predictable, precise and stable positioning in the posterior chamber of an eye with respect to the prior art. For this purpose, the invention provides a posterior chamber phakic IOL having a vault height inherent to the IOL (herein referred to as the “inherent vault height” of the IOL) which is independent from an external diameter of the IOL.

As known by the skilled person, the inherent vault height is typically a data measured along the IOL optical axis, preferably from a posterior surface of an IOL optics (i.e. a lens), typically from the intersection of this posterior surface with the optical axis, to the further (i.e. more posterior) point of IOL haptics. As known by the skilled person, the external diameter typically is a data measured perpendicularly to the IOL optical axis, and corresponds to the furthest distance between two points of the IOL.

The IOL according to the invention has therefore an axial dimension and a radial dimension independent one from the other. These two degrees of freedom allows to compensate uncertainties on expected radial compressions of the phakic IOL into an eye's posterior chamber as it dissociates axial and radial movements. As a consequence, it is possible to choose an IOL external diameter which fits to the anatomy of the eye's posterior chamber, for instance to a ciliary body-to-ciliary body distance, while providing a precise IOL vaulting with respect to the crystalline lens by another choose of IOL inherent vault height independent from the IOL external diameter, yielding then to a predictable, precise and stable positioning of the IOL in the eye's posterior chamber.

This approach differs significantly from the prior art prescribing an IOL sizing based on a single choice of IOL external diameter with an inherent vault height (and more generally, an IOL dimensioning) depending proportionally on this external diameter, limiting then the IOL sizing to a single degree of freedom solely based on uncertain radial compressions.

The phakic IOL according to invention is dimensioned to be implanted into a posterior chamber of an eye. The external diameter and the inherent vault height of the IOL can therefore be chosen based on preoperatively determined parameters to induce a predictable, precise and stable positioning of the IOL into this eye. Preferably, the external diameter is determined based (e.g. corresponds to a first function of) a white-to-white distance (WtW) of the eye. As known by the skilled person, the WtW is measured perpendicularly to the optical axis (then “horizontally” facing the eye, as a horizontal corneal measurement) between borders of the eye's corneal limbus. It is generally comprised between 11 and 13 mm. Several known technics can be used to obtain this measurement such as calipers, slit-lamp, ultrasonic biomicroscopy, magnetic resonance imaging (MRI), optical coherence tomography (OCT) and so on. The WtW is known to be easy to measure and to relate to the ciliary body-to-ciliary body (or to the sulcus-to-sulcus) distance making it possible to estimate precisely the later, and then also an appropriate IOL external diameter, based on the WtW. The first function may be expressed as WtW+ε, where ε is a correction factor preferably comprised between 0.50 and 1.00 mm, depending on other eye's anatomical parameters that can be measured preoperatively.

Equivalently, the external diameter can be directly determined based on a ciliary body-to-ciliary body (or sulcus-to-sulcus) distance measurement (in place of a WtW measurement). This measurement can e.g. be also performed by ultrasonic biomicroscopy. Although this embodiment can lead to a more precise IOL external diameter determination, the WtW may be easier to measure with more simple technics. Other equivalent measurements such as the angle to angle (AtA) and sulcus-to-sulcus (StS) distances according to the same eye dimension can fully be envisaged in the scope of the invention as it will be understood by the skilled person. The above determination of the external diameter based on WtW can then be rephrased generally as “the external diameter corresponds to a first function of a ciliary body-to-ciliary body distance and/or associated distance of the eye measured perpendicularly to an optical axis of the IOL”.

Preferably, the IOL inherent vault height is determined on basis (e.g. as corresponding to a second function) of the anterior chamber depth (ACD) of the eye. As known by the skilled person, the ACD is measured in parallel to the optical axis (i.e. “in depth ” facing the eye and/or laterally sideway) and generally comprised between 1 and 4 mm. Many known technics can be used to measure the ACD such as slit-lamp, ultrasonic biomicroscopy, Scheimpflug photography, OCT and so on. As the ACD relates to an axial distance between the cornea and the crystalline lens, it is an index of acceptable inherent vault height for the IOL (as well as preferably vault induces by the IOL above the crystalline lens once it is implanted) without the need for unreasonably relying on radial compressions exerted on the IOL once it is implanted. Other analogous, complementary and/or equivalent measurements such as the crystalline lens rise or the retroiridean space according to the same eye dimension can also fully be envisaged in the scope of the invention as it will be understood by the skilled person. The above determination of the inherent vault height based on ACD can then be rephrased more generally as “the inherent vault height corresponds to a second function of an anterior chamber depth and/or associated depth of the eye measured along the optical axis”. Considering the ACD, the second function can be expressed as a fraction of ACD, for instance about ¼ ACD. Above mentioned other eye's anatomical parameters related to the first function of WtW can comprise ACD, given it is advantageously measured and available.

According to a preferred embodiment, the phakic IOL comprises:
an anterior surface and a posterior surface;
a central optical part (or optics) comprising a lens, and extending radially relative to an optical axis directed from the anterior surface to the posterior surface;
a peripheral haptic part circumferentially mounted on the central optical part and comprising distal support elements arranged for supporting the IOL on a ciliary zonule when the IOL is in an implantation position in an eye; and
footplates mounted on the peripheral haptic part and comprising a distal border arranged for stabilizing the IOL into a ciliary body when the IOL is in the implantation position in the eye.
   In this document, the term “support element(s)” is also used as referring shortly to the “distal support element(s)”.

Preferably, the IOL peripheral haptic part extends radially outward and posteriorly relative to the central optical part of a depth depending on (preferably being) the inherent vault height. Preferably, the footplates and/or the distal support elements extends radially (beyond the proximal border of the support elements) of a length depending on the external diameter, i.e. so that to reach said external diameter. Each footplate is in particular preferably mounted on one support element of the peripheral haptic part, and extends radially outward from said support element to the IOL external diameter. Hence, the footplates extend from a most distal part of the peripheral haptic part, then reducing the footplates length for reaching the ciliary body and improving their maneuverability during an implantation process. The footplates can have a geometry (and/or radial length) independent from the external diameter prior to the IOL implantation so that to have advantageously a same predictable behavior under compression. In this case, the radial extension of the distal support elements fully vary in order for the IOL to have the external diameter. Alternatively, the distal support elements can also be designed independent from the external diameter, so that the footplates extend more or less radially from the distal support elements to reach the external diameter. Alternatively, both the footplates and the distal support elements can also have variable radial extensions depending on the external diameter.

This IOL is then equipped with two distinct and complementary haptic structures: firstly, the peripheral haptic part having the distal support elements, and secondly, the footplates. The peripheral haptic part forms together with the central optical part a “dome” (or equivalently “dome assembly”), the feet of which are the support elements arranged distally in order to support the IOL on the ciliary zonule. As the footplates extend radially beyond this dome, it also allows to stabilize the IOL into the ciliary body. The IOL is particularly stable axially (i.e. in parallel to the optical axis), radially (i.e. in perpendicular directions with respect to the optical axis) and circumferentially (i.e. in rotations around the optical axis) in implantation position.

The peripheral haptic part allows to stabilize the IOL in parallel to the optical axis. The support elements are arranged at the distal extremities of the peripheral haptic part and designed for supporting the dome on the ciliary zonule. The dome is configured to be anteriorly above the eye crystalline lens so that it encloses the lens at least anteriorly. As a consequence, the distance, called “the vault”, measured in parallel to the optical axis between the crystalline lens anterior surface and the IOL posterior surface, is defined and stabilized. The vault can be assimilated to a safety distance required in order to avoid a contact or too much proximity between the crystalline lens and the IOL. A safety distance between the IOL and the iris is similarly defined and stabilized as being the distance between the IOL anterior surface and the posterior surface of the iris conceived as a virtual iris plan occupying the pupil eye (in rise opening). Of course, both distances are depending on the IOL inherent vault height which is advantageously decoupled from the IOL external diameter and related radial compressions exerted on the IOL into the eye, and is chosen preferably based on the ACD, so that to ensure an appropriate compatibility of the IOL with the eye anatomy.

The vault is preferentially comprised and/or adjustable between 100 and 1000 μm, more preferably between 250 and 750 μm, with or without radial and/or axial compression. Enough space is then available between the IOL and both the crystalline lens and the iris, which compensates potential anatomical size defects in the eye posterior chamber, or possible positioning defects of the IOL, to sharply reduce the risks of complication for the patient. The vault can be titrated by sculpting the IOL posterior surface such that it follows a contour of a natural crystalline lens for which it is intended to be implanted.

The structure of the dome is adapted to a wide range of eye anatomy base on a choice of a posterior surface curved as an average anterior surface of any crystalline lens and on a choice of peripheral haptic part external diameter (measured perpendicularly to the optical axis), e.g. comprised between 9.50 and 11.50 mm, and compatible with a wide range of eye posterior chamber anatomy. Preferably, the dome geometry (without the feet) is globally defined by the inherent vault height as a single variable parameter. In particular, the distal support elements preferably extend from a circle around the optical axis (defining their proximal border) with a diameter (that can be called “internal diameter” in this document) independent of the external diameter of the IOL. This gives a concrete expression to the main independence of the dome with respect to IOL external diameter contrary to known phakic IOL, this contributing to a more predictable, precise and (notably axially regarding said dome) stable positioning of the IOL in the eye. Alternatively, the dome geometry can vary according to its posterior surface radius of curvature. In such case, the internal diameter can also be independent from the inherent vault height.

The dome has a posterior surface preferably smooth and (posteriorly) concave. The posterior surface at the junction between the central optical and the peripheral haptic parts preferably does not comprise irregularity or angular points. The radius of curvature of the dome posterior surface is also preferably independent of the IOL inherent vault height and of the IOL external diameter, making the dome geometry independent from the external diameter as described hereabove. Alternatively, the inherent vault height can be obtained by varying (solely) this radius of curvature between 6 and 16 mm. The radius of curvature can be chosen e.g. as a smallest average radius of curvature of an anterior surface of a crystalline lens. It is then preferably comprised between 9 and 10 mm. According to some embodiments, the dome posterior surface can comprise a relief variation at the junction between the central optical and the peripheral haptic parts, and/or the radius of curvature of the posterior surface of one or the other part can differ slightly depending on the optical requirements for the IOL.

Preferably, a thickness of the peripheral haptic part (being measured in parallel to the optical axis) is targeted to allow the IOL to reside at a determined distance from the anterior surface of the crystalline lens by selectively titrating the curvature of the IOL anterior and/or the posterior surfaces such that the posterior surface mimics the (anterior) curvature of the crystalline lens. From this, the radii of curvature of the dome anterior and posterior surfaces can also be optimized regarding the targeted dioptric power, and in a such way that the thickness at the level of the central optical part (or optics) is substantially constant across a diopter range, e.g. being of about 0.20 mm for a lens dioptric power comprised between −5 and −20 D and varying from 0.40 to 0.20 mm for lens dioptric powers varying from −0.5 to −5 D respectively. The central optical surface can be anteriorly convex and/or substantially planar and/or perpendicular to the optical axis. This allows to deliver advantageously a vault without the need for compression and therefore flexing of IOL anteriorly.

The dome tops the crystalline lens when the IOL is in an implantation position in the eye (namely, when the IOL is in a normal use in the eye), resting on the ciliary zonule, and stabilizing the IOL in parallel to the optical axis. The footplates, for their part, extend radially substantially beyond the dome in the eye posterior chamber, for laying into the ciliary body. The footplates have typically a geometry allowing to contribute to the IOL stability in its implantation position. In particular, the footplates extends radially outward from the dome so that reaching an IOL external diameter which is chosen on the basis of the posterior chamber available anatomical space for the IOL, which is preferably estimated by a WtW or ciliary body-to-ciliary body distance measurement as explained hereabove, by taking preferably into account an elasticity penetration factor of the footplates in the eye ciliary body. This advantageously limits radial compressions exerted on the footplates and their impact on the vault (or IOL axial positioning). Moreover, the flexibility of the footplates can contribute to the IOL adaptability to internal size variations of the posterior chamber anatomical space. As the dome dimensions remain substantially constant for an inherent vault height, the external diameter of the IOL can then vary at the footplates and/or distal support elements level.

The footplates further enable the stabilization of the IOL in rotation in a plane perpendicular to the optical axis so that the variation in this anatomical space (acknowledged to be greater in one orientation than another because of its “oval” shape) can be fully compensated. These footplates are more preferably circumferentially and radially extended such that their distal borders are adjusted and/or configured to lay and/or hook and/or stabilize themselves into the ciliary body, playing a role of circumferential anchors for the IOL. This is important in the embodiment for which the IOL is a toric implant comprising an optic with a cylinder to correct astigmatism. Indeed, in this case, the stability of the angular position of the IOL lens in the perpendicular plane, so called the “rotational stability”, allows to guarantee the expected IOL optical results. The footplates allow to maintain the IOL lens in an expected optical zone and then to avoid possible decentering of the IOL with respect to the eye optical axis which could affect the IOL optical results.

Each of the footplates can be of any kind according to the prior art, including a massive distal footplate, a long thin freely ended flexible haptic or a flexible haptic elongated from a first extremity to a second extremity both directly mounted on one of the support elements. This last case is however preferred and the distal border of a footplate is therefore preferably a “distal lateral border” of the elongated flexible haptic. The fixation of the first and second extremities on the support element allows the footplate to extend from and to a most distal part of the peripheral haptic part, then reducing the footplates length for reaching the ciliary body and improving their maneuverability during an implantation process.

The first and second extremities are typically the footplate's only part mounted on the IOL peripheral haptic part. More generally, the footplates are not mounted over their (entire) length on the peripheral haptic part. As the first and second extremities of each footplate are mounted on a support element, the footplate extends globally along a partial loop reaching so a distal extremity of the loop at the IOL external diameter. The distal border has then a form of a portion of this partial loop arranged distally to stabilize the IOL into the ciliary body. The distal border advantageously proposes a potentially wide contact surface with the ciliary body.

The “elongated” feature of the haptics forming the footplates refers to a preferred thin geometry, which contributes to its flexibility as described above. Each footplate has preferably three dimensions among which its length along a main trajectory of extension, its thickness, and a width measured orthogonally to the two other dimensions. This “elongated” feature can be translated by a length (at least twice or at least three times) greater than the (average) thickness and the (average) width of the footplate. This provides to the footplate a great ability to deform under axial and/or radial compressions of the IOL, contributing to more stability adaptability to of the IOL to the anatomy of the eye posterior chamber.

The footplates preferably borders (preferably open) cavities extending from the anterior to the posterior surfaces of the IOL. Preferably, each cavity is completely bordered by a footplate and one of the support elements on which it is mounted. The term “cavity” is here used as an equivalent to a space empty of the material constituting the IOL. The cavities are preferably not holes made in said material but simply a feature arising from the footplates geometry.

Each footplate can optionally comprise a material fold and/or a lateral recess, e.g. at its extremities, arranged to facilitate and/or to direct the footplate curvature and/or orientation when axial and/or radial pressure is exerted on the IOL. In particular, such material fold and/or lateral recess can play the role of a failsafe mechanism arranged to prevent an excessive force transmission from the footplates to the central optical part.

The distal border of each footplate optionally comprises smooth ripples arranged to smoothly hook into the eye ciliary body. Advantageously, the ripples facilitate the stabilization of the IOL into a ciliary body when it is in the implantation position in the eye. The ripples give a role of a pin to the distal border to lay and stabilize easier into a ciliary body. These ripples are preferably smooth so that their contours cannot irritate the ciliary body or other part of the eye anatomy.

The IOL and, specifically, the peripheral haptic part and the footplates are preferably made of a biocompatible, flexible and highly resistant material. This material is preferably a hydrophilic material. The “rigid” structure of the dome is typically induced by a greater thickness (measured in parallel to the optical axis) in material in average than that of the footplates, and/or by a flared and/or wide and/or thick shape of the support elements. In contrast, the “flexible elongated” feature related to haptics of the preferred footplates embodiment is induced by the nature of the material combined with an elongated geometry as well as a low average thickness, notably in comparison with that of the dome. In this document, the term “flexible” referring to the haptics may however be omitted as being more qualitatively induced by the above described haptics geometry.

The two haptic structures described constitute a compromise between on one hand, the need for stability and compliance with intraocular structures, and on the other hand, the need for rigidity, avoiding excessive exertion of force and trauma to delicate intraocular structures, many of which are complex and not visible during or prior to implantation. They are structured to ensure that the vault as described above is not significantly affected from compressions exerted on the edges of the IOL by the eye internal anatomy.

This structure follows from the independence of the IOL inherent vault height from the IOL external diameter, this allowing to choose both parameters as being the most appropriate to eye internal anatomy (e.g. ACD and ciliary body-to-ciliary body distance as above) limiting then such compressions and impacts on the vault. In particular, the double degree of freedom allows to minimize this while ensuring a better harmonization of the compressions for each IOL sizing.

In the framework of the present document, an “optical axis” of an eye consists preferentially in a vector crossing the eye from one side to the other, directed by its “anterior segment”, comprising successively, the cornea, the iris and the lens, to its “posterior segment”, comprising the retina. For a phakic IOL according to the invention in an implantation position in an eye, the optical axis of the eye is directed from the IOL anterior surface to the IOL posterior surface and preferably corresponds to the optical axis defined intrinsically with respect to the IOL. In particular, the term “optical axis” is preferably used in the present document as the reference axis with respect to the eye and/or to the IOL.

In the framework of this document, an “anterior” (or resp. “posterior”) side and/or surface of an eye or IOL part or consists preferably in a side and/or surface located upstream (or resp. downstream) of said part with respect to the vector defined by the optical axis. This definition extends naturally to the term “anteriorly” (or resp. “posteriorly”). By the way of examples, in an eye, the iris is located anteriorly with respect to the crystalline lens; a posterior surface of the iris is then a part of the iris that is the closest to the crystalline lens.

Likewise, such side and/or surface is said “anteriorly concave” (resp. “anteriorly convex”) when it is seen as concave (resp. convex) by looking at the optical surface in the same direction and the same sense as the vector defined by the optical axis (i.e. following light rays'propagations). It is said “posteriorly concave” (resp. “posteriorly convex”) when it is seen as concave (resp. convex) by looking at the optical surface in the same direction and the opposite sense as the vector defined by the optical axis. In this document, the term “concave” is generally used as corresponding to “posteriorly concave” when the context of its use make it clear for the skilled person that this is its meaning.

The aforementioned notions of anteriority, posteriority or even of an optical axis relative to parts of an eye and/or of an IOL are well known to person skilled in the art. In particular, the IOL according to the invention is configured to be positioned in the posterior chamber of an eye, so that its anterior surface is at least partially facing the iris of the eye and so that its posterior surface is at least partially facing the crystalline lens of the eye.

In the framework of this document, the term “axial” and “axially” refer to directions in parallel to the optical axis. It is preferably said that part of an IOL extends:
“radially” if it extends according to vectors perpendicular to the optical axis,
“radially outward” if the vectors are directed from a point in common with the optical axis to points of a circle centered at this common point; and
“radially inward” if the vectors are directed in the opposite sense.
   It is also preferably said that a part of an IOL extends “circumferentially” when it extends preferably according to a circular arc on a plane perpendicular to the optical axis centered on an intersection point of this plane and the optical axis. These notions of radial and circumferential extensions refer to known systems of polar coordinates in each plane perpendicular to the optical axis.

It is well known by a person skilled in the art that the adjective “distal” refers to a part of a portion of a body the furthest form a reference organ or from a trunk of a body, and that the adjective “proximal” refers to a portion of a part of a body the closest to a reference organ or to a trunk of a body. In the framework of this document, these definitions also apply to parts of an eye and/or of an IOL, relative to a distance with respect to the referential optical axis. By the way of examples, preferably, a proximal portion of an IOL according to the embodiment above mentioned may comprise the central optical part and/or a part of the IOL around a central part, and a distal portion of an IOL according to the invention may comprise the footplates, or at least their distal borders. In particular, the term “distal” concerning the “distal (lateral) border” of a footplate refers preferentially to the set of points of the footplate each being the furthest from the optical axis along a radius perpendicular to the optical axis.

In the framework of this document, the use of the verbs “comprise”, “have”, “include” or variants, does not exclude the presence of other elements than those mentioned. The use in this document of the terms “a”, “an” or “the”, to introduce an element also does not exclude the presence of several of these elements. Also, in this regard, the present application may use the term “plurality” to reference a quantity or number. In this regard, the term “plurality” is meant to be any number that is more than one, such as two, three, four, five, etc. Unless otherwise indicated, the terms “about”, “approximately” or variants, in reference to a value, means that the value is provided with an error margin of 10%.

In this document, the terms “first”, “second”, “third”, and the like are used for distinguishing similar elements without necessarily inducing sequential or chronological order, unless otherwise indicated. In this document, the steps of a method are preferably executed in their presentation and/or numbering order.

For limiting manipulations needed to position the IOL between the eye crystalline lens and the iris and making easier the footplates maneuverability during the implantation process, the peripheral haptic part is preferably endowed with a manipulation pocket on the IOL anterior surface, radially aligned with one of the footplates. The pocket is preferably dimensioned for cooperating with a tip of a manipulation tool by a keyed engagement of the tip into the pocket, so that a moving of the footplate can be induced by a moving of the tool. The pocket is typically arranged close to the footplate as being radially aligned with it.

Advantageously, no direct manipulation of the footplates is necessary which strongly reduces the risk of manipulation error. Indeed, as the footplates can be thin and clear, it may be easy to miss out, to pass through the IOL posterior surface and to touch sensible intraocular tissues, such as the crystalline lens. By circumscribing the tip movement in such a pocket at level of the sole IOL anterior surface, no such handling error can occur. The pocket is advantageous when the IOL has to be manipulate under the eye iris which is opaque. Indeed, it allows to guide the tip of the tool on the anterior surface by a keyed engagement of the tip into the pocket. So, it is not necessary for the surgeon to see the footplate under the iris for positioning it correctly. It is advantageously enough for the surgeon to perform appropriate (known) movements with the tool, when the tip is engaged in the pocket, for positioning adequately the footplate, without necessarily seeing it, as described in detail in the document WO 2023/021225 A2, the content of which is integrated herein by reference. In particular, the tool that can be used is described in detail in this document, as well as the corresponding movements to be performed during the implantation process. With this respect, the pocket is preferably especially dimensioned for fitting with these movements and guiding the surgeon during the implantation process. An extreme slipperiness of the IOL material design makes the IOL safe and painless for the eye intraocular tissues and manipulation difficulties that can arise therefrom is fully compensate by the presence of manipulation pockets.

Preferably, the pocket is arranged (at least partially) on (the anterior surface of) a support element to which the footplate is mounted when it applies. The pocket can however also slightly extend partially on the footplate itself. Each footplate is preferably associated with such a (more preferably specific) pocket as mentioned and the herein described embodiments relating to “the pocket” can apply to all the pockets. The number of pockets can correspond to the number of footplates, each pocket being dedicated to the manipulation of a single footplate.

According to a preferred embodiment of the invention, the inherent vault height of the IOL is chosen among N>1 possible values, and the external diameter of the IOL is chosen among M>1 other possible values. These N and M values are preferably chosen in order to cover adequately in average all eye anatomies as described above, providing then only N+M IOL models to manufacture and a two degree of freedom for the surgeon to choose the appropriate IOL to be implanted into a specific eye. It can then be provided in the framework of the invention a set of such posterior chamber phakic IOLs, comprising (preferably consisting of) N×M IOLs, all having distinct pair of inherent vault height and external diameter. In the set, two IOLs can then have the same external diameter but a different inherent vault height, ensuring to offering two independent parameters for a more predictable, precise and stable positioning of each IOL in a corresponding eye's posterior chamber.

In particular, the present invention provides a method for choosing a IOL to be implanted into a posterior chamber of an eye among such a set of IOLs, the method comprising:
a first step of:
   measuring the WtW of the eye;
   selecting the external diameter of the IOL as the closest value to a first function of the measured WtW among said M possible values;
a second step of:
   measuring the ACD of the eye;
   selecting the inherent vault height of the IOL as the closest value to a second function of the measured ACD among said N possible values;
and a third step of:
   choosing the IOL among the set having the selected inherent vault height and external diameter.

The description and advantages regarding the preceding embodiment for which the IOL external diameter (resp. inherent vault height) is determined based on WtW (resp. ACD) apply to this method. The latter allows to choose a phakic IOL according to invention adequately dimensioned to be implanted into a posterior chamber of an eye based on said preoperatively determined parameters so as to induce a predictable, precise and stable positioning of the IOL, without the need for manufacturing specifically the IOL according to these parameters. The above embodiment is not limited to the WtW measurement and extends to equivalent or analogous distances measurement perpendicularly to the IOL optical axis, e.g. the ciliary body-to-ciliary body, as explained hereabove. This embodiment is not limited to the ACD measurement and extends to other equivalent or analogous depth measurement along the IOL optical axis, e.g. the crystalline lens rise, as explained hereabove.

It has been discovered that M =3 or 4, N=2, 3 or 4, provide enough IOLs in the set to reach this technical effect. The IOL external diameter is preferably comprised between 11.50 and 14.50 mm while the inherent vault height is preferably comprised between 1.00 and 2.00 mm when no compression is exerted on the IOL. It is preferably proposed M=3 values these being about 12.70 mm, 13.20 mm and 13.60 mm or more preferably M=4 values these being about 11.70 mm, 12.50 mm, 13.30 mm and 14.10 mm, each then covering a wide class of eye's posterior chamber anatomy. It is preferably proposed N=3 values, corresponding to three dome heights, “small”, “medium” or “large”, these being about 1.15 mm, 1.45 mm and 1.75 mm, each adapted to a wide class of ACD.

Another object of the invention is to provide a manufacturing method of a phakic IOL to be implanted into an eye's posterior chamber inducing a more predictable, precise and stable positioning of the IOL in the eye. To this end, it is provided such a method comprising:
(i) determining an external diameter of the IOL;
(ii) determining a vault height inherent to the IOL independently from the external diameter;
(iii) manufacturing the IOL having the external diameter and the inherent vault height.
   The method therefore provides a phakic IOL having a vault height inherent to the IOL independent from an external diameter of the IOL, so that the embodiments and the advantages of the IOL according to the invention as described herein are transposed mutatis mutandis to the present method.

In particular, preferably, the IOL is dimensioned to be implanted into a posterior chamber of an eye, and the manufacturing method comprises:
measuring a WtW the eye before the step (i), and
measuring an ACD of the eye before the step (ii).
   The external diameter is then determined at step (i) depending on the measured WtW and the inherent vault height is determined at step (ii) depending on the measured ACD. This embodiment is not limited to the WtW measurement and extends to equivalent or analogous distances measurement perpendicularly to the IOL optical axis, e.g. the ciliary body-to-ciliary body, as explained hereabove. This embodiment is not limited to the ACD measurement and extends to other equivalent or analogous depth measurement along the IOL optical axis, e.g. the crystalline lens rise, as explained hereabove.

According to a preferred embodiment, as before, the IOL to manufacture comprises:
an anterior surface and a posterior surface;
a central optical part (or optics) comprising a lens, and extending radially relative to an optical axis directed from the anterior surface to the posterior surface;
a peripheral haptic part circumferentially mounted on the central optical part and comprising distal support elements arranged for supporting the IOL on a ciliary zonule when the IOL is in an implantation position in an eye; and
footplates mounted on the peripheral haptic part, preferably on the support elements, and comprising a distal border arranged for stabilizing the IOL into a ciliary body when the IOL is in the implantation position in the eye.
   In this case, the manufacturing method preferably comprises:
between the steps (i) and (iii), designing the footplates and/or the support elements as extending radially (beyond the proximal border of the support elements) of a length depending on the external diameter; and
between the steps (ii) and (iii), designing the peripheral haptic part as extending radially outward and posteriorly relative to the central optical part of a depth depending on the inherent vault height.
   All the embodiments and corresponding advantages related to this preferred IOL according to the invention described above also extends mutatis mutandis to this preferred embodiment of the method. In this document, the peripheral haptic part is preferably constituted by a main proximal portion (defining mainly the dome) to which is mounted distally the distal support elements. The footplates and/or the distal support elements are then designed as above between steps (i) and (iii) as extending radially beyond the main proximal portion of a length depending on the external diameter. The same applies to the corresponding embodiment of the IOL object of the invention. The terms “proximal border of the distal support elements” can also be replaced by “a junction curve (being typically a circle) between the distal support elements and the main proximal portion.

According to an embodiment of the manufacturing method according to the invention, it further comprises between the steps (ii) and (iii), designing optical surfaces of the IOL depending on refractive efficiencies to be achieved, the IOL being manufactured at step (iii) as comprising these optical surfaces. In particular, the optical surfaces as being the restriction of the anterior and posterior surfaces of the IOL at the central optical part (or lens) level can be optimized regarding a targeted dioptric power and based on a crystalline surface to mimic as explained herein before.

This allows a design of a lens of the central optical part that is best adapted to a defect in vision to be corrected. For instance, it can be a monofocal lens allowing a correction of myopia, hypermetropia, presbyopia and/or corneal astigmatism. The lens can be refractive or diffractive, and/or at extended depth of focus, in order to treat presbyopia. The lens can be selected according to the state of the art.

The disclosed subject matter is further introduced in the claims. As it will be easily understood by a skilled person from the disclosure of the invention, the dependency of the claims can be considered in a broader manner so that any possible combination of the claims—as far as they are technically possible and understood by the person skilled in the art in view of the present disclosure - are part of the present disclosure.

### BRIEF DESCRIPTION OF THE FIGURES

Other features and advantages of the invention will appear on reading the following detailed description, for the understanding of which, reference to the attached drawings is made. The drawings are incorporated in the FIGS. 1 to 8 as introduced below:

FIG. 1 illustrates a global tridimensional anterior and lateral view of an IOL according to a preferred embodiment of the invention;

FIG. 1A illustrates a global tridimensional anterior and lateral view of an IOL

FIG. 2 illustrates a planar top view of the IOL illustrated in FIG. 1;

FIG. 3 illustrates a side shadow view of the IOL illustrated in FIG. 1 with a relative positioning of an IOL induced dome and an eye crystalline lens;

FIG. 4 illustrates a tridimensional enlarged view of a section of a support element and a footplate of the IOL illustrated in FIG. 1

FIG. 5 illustrates a sectional view of part of an eye wherein is fitted the IOL illustrated in FIG. 1, the latter being illustrated by a side shadow view;

FIG. 6 illustrates interferences between radial compressions into the eye and axial positioning an IOL according to the invention on part of FIG. 5;

FIG. 7 illustrates an execution of the manufacturing method according to the invention on side shadow views of the IOL to manufacture;

FIG. 8 illustrates the independence of the vault height inherent to an IOL of the invention from its external diameter on a side shadow view of the IOL.

The drawings are typically not scaled. Similar elements are generally assigned by similar references. In this document, identical or analogous elements may have the same references. Reference in the drawings cannot be considered to be limiting, comprising when these references are indicated in the claims.

### DETAILED DESCRIPTION

This part of this document presents a full description of specific and preferred embodiments of the present invention with references to the drawings. The invention is however not limited by these references. The Figures that were introduced above are in particular only schematic and not limiting in any way.

Some of the Figures are provided with abstract geometric marks and corresponding references substantially used to quantify and/or visualize technical characteristics of embodiments of the invention such as measures or geometric characteristics. These geometric marks generally do not correspond to concrete material objects.

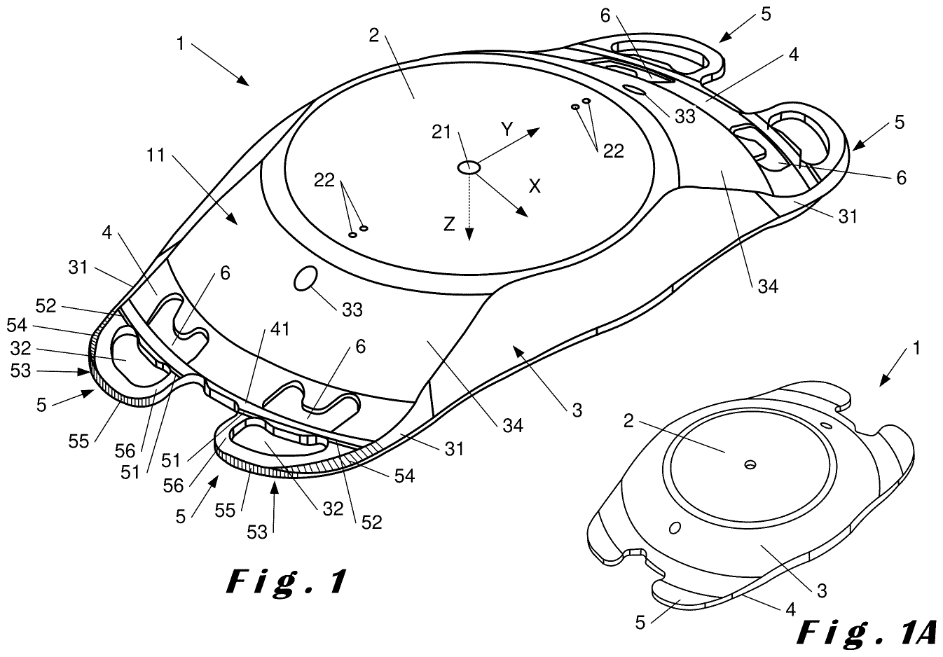

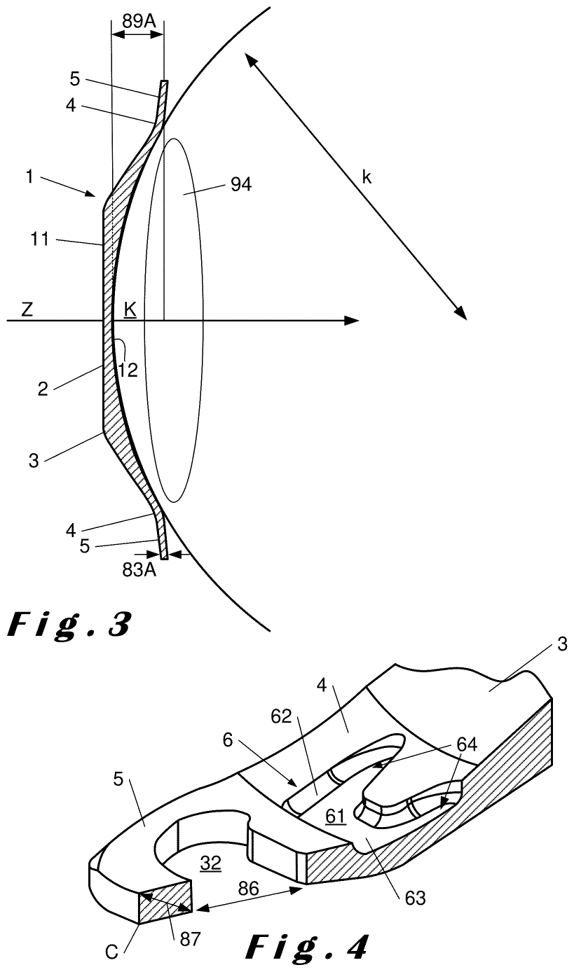

The invention provides a posterior chamber phakic IOL adapted to a broad range of eye anatomies, easy to implant and postoperatively stable in an implantation position in an eye 9, axially along an optical axis Z, radially and in rotation in a plane perpendicular to the optical axis Z based on vectors (or axis) X and Y. In particular, as illustrated in FIG. 1, the axis X, Y and Z form an orthogonal basis of the Euclidian tridimensional space. As conventionally, the optical axis Z is directed from an anterior surface 11 to a posterior surface 12 (referenced on FIGS. 1 and 3) of the IOL 1.

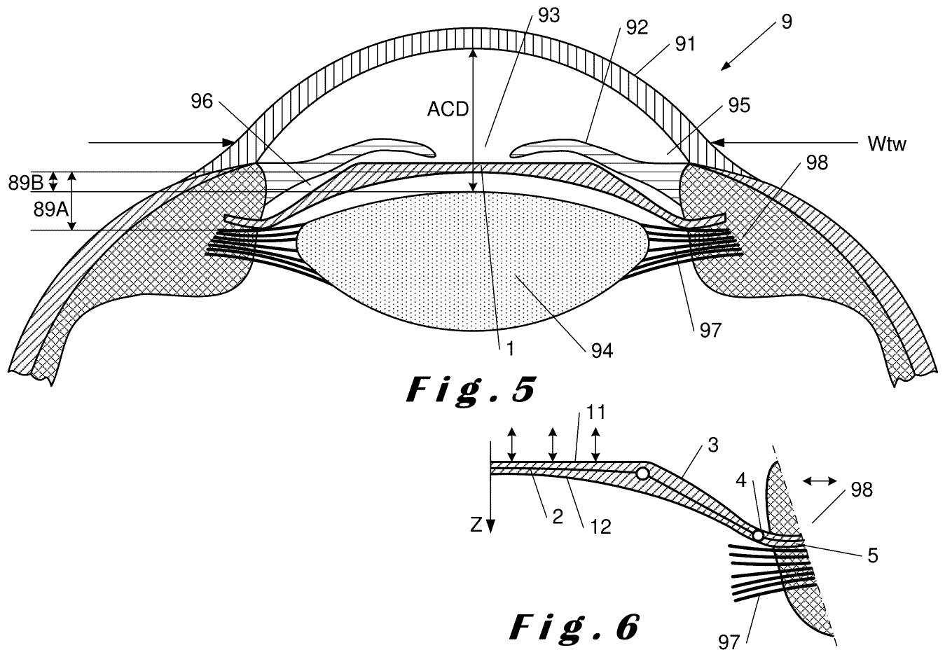

As represented on FIG. 5, the IOL 1 is intended to be positioned in the posterior chamber 96 of an eye 9. Other elements of the eye 9 anatomy are illustrated on FIG. 5: a cornea 91, an iris 92, a pupil 93, a crystalline lens 94, an anterior chamber 95, a ciliary zonule 97 and a ciliary body 98 of the eye 9. The white-to-white distance is represented by WtW, and the anterior chamber depth by ACD (measured prior to the IOL 1 implantation).

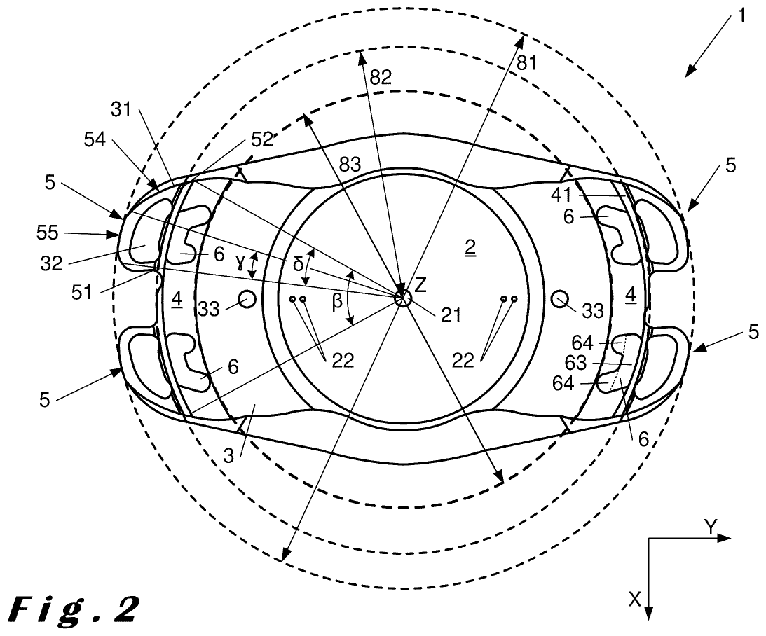

As illustrated in FIGS. 1 and 2, the IOL 1 has a central optical part 2 extending radially relative to the optical axis Z on a maximal external diameter comprised between 4.5 and 6.7 mm, preferably of about 5.8 mm. It comprises a through bore 21 extending along the optical axis Z between the anterior 11 and posterior 12 surfaces, so that a fluid communication is possible between these surfaces. The central optical part 2 also comprises orientation marks 22 in form of two pair of diametrically opposed tiny superficial (non through) holes aligned along axis Y on the anterior surface 11. Those marks 22 can be used for orienting the IOL 1 during its implantation, which is in particular useful for toric IOL 1. The orientation mark 22 can take various other forms such as at least one superficial line made by laser, mild, or engraving on the anterior surface 11 of the IOL 1. Exemplary diameters values of the through bore 21 and each superficial hole are respectively about 0.36 and 0.12 mm. Other orientation marks (not shown) such as small lateral bars on the peripheral haptic part 3 can be used to avoid upside-down implantation.

The central optical part 2 is surrounded by haptic structures among which a peripheral haptic part 3 circumferentially and proximally mounted on the central optical part 2. The peripheral haptic part 3 extends radially outward and posteriorly relative to the central optical part 2 of a depth measured along the optical axis Z depending on a desired IOL 1 inherent vault height 89A illustrated on FIGS. 3 and 5. The latter is determined prior to the IOL 1 implantation based on the ACD as explained in the disclosure. The peripheral haptic part 3 extends further radially along axis Y than along axis X, so that the IOL has a global planar form elongated along axis Y as illustrated on FIG. 2.

The peripheral haptic part 3 is composed of a main proximal portion 34 and two diametrically opposed distal support elements 4. The main proximal portion 34 comprises two peri-optical holes 33 arranged proximally close to the boundary with central optical part 2, along axis Y, symmetrically with respect to the optical axis Z. The peri-optical holes 33 cross the IOL 1 through the anterior 11 and the posterior 12 surfaces so that they allow a fluid flow during the IOL 1 implantation process. As represented on FIG. 2, the peri-optical holes 33 are comprised in a IOL 1 part centered on the optical axis Z of a diameter comprised between 7.2 to 8.0 mm, preferably of about 7.45 mm. The peri-optical holes 33 are preferably quite similar to the through bore 21 in terms of size.

The support elements 4 are attached on diametrically opposed distal extremities of main proximal portion 34, in mirror symmetry with respect to a plane based on axis X and on the optical axis Z. They have the form of a ring portion extending circumferentially around the optical axis Z, each along an arc of circle with a central angle β between 60° and 80°, e.g. of about 70° (visible on FIG. 2). As it is known, the term “central angle” refers generally to the angle subtended by the circular arc. In particular, it is the angle at a center of circle of the circular arc of a triangle whose vertex are said center and the two extremities of the circular arc. An (distally) extremal border 41 of each support element 4 extends in particular along an arc of circle of a radius 82 comprised between 4.5 and 5.8 mm, the value of which can depend on the IOL external diameter 81.

In particular, the whole peripheral haptic part 3 and the central optical part 2 are inscribed in a cylinder of the radius 82 which extends around the optical axis Z, so to form a dome K (or dome assembly) with a desired inherent vault height 89A which is supported posteriorly by the support elements 4. Also, the main proximal portion 34 and the central optical part 2 are inscribed in a cylinder of a diameter 83 (that can be also called “internal diameter” in the framework of this document) which extends around the optical axis Z, so to form the dome K (without the feet) with a desired inherent vault height 89A. This diameter 83 is independent from the IOL external diameter 81 and can have e.g. three values of about 9.0, 10.2 and 11.3 mm, respectively for the three dome heights (or domes) herein described. As shown on FIGS. 3 and 5, the dome K has a posterior surface to top a crystalline lens 94 when the IOL 1 is in its implantation position in an eye 9. The distal support elements 4 are then arranged for supporting the IOL 1 on a ciliary zonule 97 of the eye 9.

The dome K posterior surface is concave, smooth, and curved with a preferred radius k of curvature of about 10 mm compatible with the curvature of the crystalline lens 94 anterior surface, so that a vault 89B adjustable between 250 and 750 μm can be ensured between the IOL 1 and the crystalline lens 94 when the IOL 1 is in its implantation position as shown on FIG. 5.

A “wall thickness” confers rigidity to the dome K so that it is resistant under axial and/or radial compression. The thickness of the dome K around the optical axis Z may be about 0.20 mm, then it increases radially till reaching the proximal boundary of the peripheral haptic part 3, having for instance a thickness of about 0.60 mm, and finally decreases radially till the support elements 4 having a thickness generally comprised between 0.15 to 0.30 mm (without taking into account the pockets introduced hereafter). These values are selected so that the dome K is able to constitute a sufficiently rigid and broad structure to surround and top anteriorly a crystalline lens 94, and thereby to be implanted in a broad range of eye anatomies, while being stable in parallel to the optical axis Z.

As illustrated clearly in FIGS. 1, 2 and 4, the IOL 1 also comprises two pair of diametrically opposed footplates 5 as flexible haptics each elongated from a first extremity 51 to a second extremity 52 both directly mounted on one of the distal support elements 4. The footplates extend radially along axis Y from and beyond the peripheral haptic part 3 of a length which can depend or not on a desired external diameter 81 of the IOL 1 and depends on radial compression exerted on the IOL 1. The external diameter 81 is determined prior to the IOL 1 implantation based on the WtW or on a ciliary body-to-ciliary body (or equivalent) measurement as explained in the disclosure of the invention, so that it is well adapted to the eye 9 anatomy. As shown on FIG. 2, the extremal border 41 extends between the first 51 and the second 52 extremities along a circular arc of the radius 82 with a central angle δ comprised between 15° and 45°, e.g. of about 20° to 25°. Each footplate 5 has a form of a partial loop bordering a cavity 32 extending from the anterior 11 to the posterior 12 surfaces.

Each cavity 32 is more extended in terms of area perpendicularly to the optical axis Z than the corresponding footplate 5. In particular, as shown on FIG. 4, a maximal radial length 86 of each cavity 32 is (much) greater than a maximal diameter 87 of any cross section C of the footplate 5. This radial length 86 is comprised between 0.60 and 0.90 mm, preferably of about 0.70 mm, and the radial length of said cross section C is preferably comprised between 0.20 and 0.40 mm. The surface of the IOL 1 extending radially further than the circle of radius 82 is therefore emptier of a solid matter that full of a solid matter. Each footplate 5 can have a constant thickness 83A (shown on FIG. 3) comprised between 0.10 and 0.30 mm, preferably of about 0.17 mm. These data contributes to give a great flexibility to the footplates 5.

The IOL 1 is inscribed in a cylinder of the external diameter 81 with a preferred value between 11.5 and 14.5 mm prior to implantation, when no axial or radial compression is exerted on the IOL 1. Each footplate 5 extends between the circle of radius 82 and the circle of external diameter 81, so that its flexibility allows to compensate size variations of an anatomical space available in the eye posterior chamber 96 for the IOL 1 when the latter is in its implantation position without significantly affecting the dome K positioning as explained above in the disclosure of the invention. With this respect, the inherent vault height 89A and the external diameter 81 are independent one from the other for ensuring a more predictable, precise and stable positioning of the IOL 1.

Indeed, this independence avoids targeting the positioning of the IOL 1 along the optical axis Z based on an expected radial compression on the IOL 1 into the eye 9 which lead to errors and unstable positioning. Such positioning is shown on FIG. 6 with double arrows and fictive hinges on junctions between the central optical part 2 and the peripheral haptic part 3, and between the latter and the footplates 5: when a radial compression is exerted toward the IOL 1 (left arrow), the IOL 1 structures (schematized by said hinges) induces a pushing of the central optical part anteriorly (up arrow), and reversely. By targeting the IOL 1 inherent vault height 89A (and consecutively the vault 89B) independently from the external diameter 81, each of these parameters is more adapted to the eye 9 anatomy for respective axial positioning (by the dome K) and radial/rotational positioning (by the footplates 5) of the IOL 1 as detailed hereabove, so that the axial positioning of the IOL 1 is independent or at least strongly less dependent on radial compressions on the footplates 5 according to the mechanism shown on FIG. 6.

Each footplate 5 is specially designed for folding and/or curving when compression is exerted axially and/or radially on IOL 1, in such a way that an adjustable angle between the optical axis Z and a normal vector to a plane of extension of the footplate 5 is generally comprised between −15° and 15°. Each footplate 5 comprises a distal border 53 extending circumferentially and radially outward relative to the support element 4 on which it is mounted. This distal border 53 is in particular arranged for stabilizing the IOL 1 into the ciliary body 98 when the IOL 1 is in its implantation position as illustrated on FIG. 5. It acts as an anchor for stabilizing the IOL 1 in rotation in a plane perpendicular to the optical axis Z as detailed in the disclosure of the invention. The distal border 53 may optionally be arranged for stabilizing the IOL 1 into the ciliary sulcus of the eye 9, or more generally within a corresponding eye structure (taking into account possible practical position variation in the footplates during IOL implantation), so that the terms “ciliary body” in the present document could optionally be replaced by “ciliary body and/or sulcus”.

The distal border 53 is composed of a first 54 and a second 55 portions particularly visible on FIG. 1. As represented on FIG. 2, the second portion 55 extends along a circular arc of the external diameter 81 with a central angle γ comprised between 7.5° to 20°, and typically of about 10° when no axial or radial compression is exerted on the IOL 1. It consists therefore in the most distal part of the distal border 53. The first portion 54, for its part, extends from the second extremity 52 to the second portion 55.

The IOL 1 has a smooth lateral chamfer 31 extending smoothly and continuously, from a support element 4 on which is mounted a footplate 5, to the first portion 54 of the distal border 53. This chamfer 31 extends on the support element 4 and on the first portion 54 of the distal border 53, so that as providing a continuous and smooth lateral transition between the peripheral haptic part 3 and footplates 5 via one of the footplate extremities, e.g. the second extremity 52. This transition is particularly helpful for implanting the IOL 1 because it allows to insert smoothly the footplates 5 under the eye iris 92. Optionally, the chamfer 31 can extend on all the distal border 53 (not shown).

Each footplate 5 may consist substantially in three portions: a natural first width extension of the first portion 54, a natural second width extension of the second portion 55, and a third portion 56 visible on FIG. 1 connecting the natural second width extension and the first extremity 51. The main extension trajectory of these footplate portions extends respectively both circumferentially and radially, substantially only circumferentially and substantially only radially in a direction having a smaller angle with axis Y (corresponding typically to (½)(β-2δ)) comprised between 5 and 60°, e.g. of about 7.5° in the case of the illustrated embodiment of the invention. This angle allows advantageously to decrease the exerted compression forces on the IOL 1 when it is in its implantation position. In particular, a higher angle than 7.5°, such as 10°, 12.5°, 15°, 17.5°, 20°, 25°, 30° or 40° is also preferred as the greater it is, the lower are the exerted compression forces on the IOL 1.

The global design of the footplates 5 is determined to facilitate the IOL 1 implantation process. In particular, the chamfer 31 can have a concave smooth external surface so that each footplate 5 is distally oriented in a convergent way toward the axis Y. Movements to insert the footplates 5 under the iris 92 are then greatly easier. The extremal border 41 of each distal support element 4 extends further between the first extremities 51 of footplates 5 of two different pairs along a circular arc of the radius 82 with a central angle of about 15 to 20°.

The illustrated embodiment of the IOL 1 has two diametrically opposed support elements 4 and four footplates 5, symmetrically arranged, for ensuring a good stability of the IOL under axial and/or radial compressions, as well as in rotation. In particular, when a compression lateral force occurs, the IOL 1 design absorbs efficiently the latter partially within the footplates 5 arranged according to a predetermined external diameter 81, so that the dome K is advantageously axially stable with an inherent vault height 89A dedicated to the eye 9 anatomy. The use of four footplates 5 symmetrically arranged (e.g. on the corners of a rectangle) is further preferred because this mitigates any possible tilting effect. However, the scope of the invention is not limited to these specific numbers and arrangements of support elements 4 and footplates 5.

Given that the footplates 5 are particularly flexible, it is advantageous to provide the IOL 1 with a structure for helping to control the movements of the footplates 5 during the implantation process and to insert them under the iris 92. To this end, the support elements 4 can comprise manipulation pockets 6 on the IOL anterior surface 11 seen on FIGS. 1, 2 and 4. Each pocket 6 is associated with a footplate 5 structurally and functionally. In particular, structurally speaking, each pocket 6 faces the associated footplate 5, so that only the extremal border 41 separates the cavity 32 from the pocket 6. Each pocket 6 is also radially aligned between the first 51 and second 52 extremities of the corresponding footplate 5, which improve further the footplate 5 manipulation by an engagement of the tool tip into the pocket 6. The pockets 6 remain however optional and the scope of the invention is not limited to their presence or form. A circumferential trench 63 can be defined therefrom on the IOL anterior surface 11 extending in parallel to the footplate 5 and comprising radially inwards extensions 64 arranged at two circumferential extremities of the trench 63, in mirror symmetries with the footplate extremities 51 and 52.

The trench 63 has a rough bottom surface 61 and lateral edges 62 of an axial height of about half of a thickness of the corresponding support element 4. The corresponding axial height is comprised between 0.075 and 0.125 mm, preferably between 0.08 and 0.09 mm. A most distal lateral edge, at the boundary with the extremal border 41, may be shaped in a half cylindrical form with radius 0.06 mm. Such a pocket is easy to manufacture and is fully satisfactory for the described manipulation purpose. These geometrical features of the pockets 6 are provided in order to allow a functional cooperation with a tip of a manipulation tool by a geometrical keyed engagement of the tip into the pocket 6, so appropriate moving of the footplate 5 during the implantation process of the IOL 1 can be induced by a moving of the tool as explained in detailed in WO 2023/021225 A2.

As mentioned hereabove, the invention is not limited to the footplates 5 of FIG. 1 and to the presence of manipulation pockets 6. For instance, FIG. 1A illustrates another embodiment of the IOL 1 according to the invention, where the footplates 5 are massive footplates, without cavities 32, and without pockets 6, which is fully part of the scope of the invention.

An embodiment of the phakic IOL 1 manufacturing method according to the invention is represented on FIG. 7. Continuous lines represent part of the IOL 1 designed at a pending step (A), (B) or (C) of the method, before the IOL 1 to be manufacture afterwards. Dotted lines represent other part of the IOL 1. For each step, three design options for the IOL 1 part designed at the step are shown but more or less can be considered. In this sense, although it is herein referred to as a manufacturing method according to the invention, this can also be seen as an execution of the IOL choosing method according to the invention (wherein M=N=3, for said three design options), (A) being the first step, (B) the second step, and (C) being an extra step of choosing an appropriate optics or lens. Steps (A), (B) and (C) can also be referred to as a “design method” of the IOL 1 prior to its manufacturing in the framework of this document.

At step (A), the “best” external diameter 81 of the IOL 1 is determined. This can be done on the basis of the WtW of the eye 9 preoperatively measured in the X-Y plane (i.e. horizontally), or on the basis of other measures as explained in the disclosure of the invention. Based on step (B), the footplates 5 and the support elements 4 will then be designed as extending radially from and beyond the distal extremity of the main proximal portion 34 of the peripheral haptic part 3 (or equivalently from and beyond the support elements 4 proximal border, i.e. from and beyond a cylinder extending around the optical axis Z with a diameter 83 according to the dome geometry set at step (B)), to the external diameter 81. For instance, the support elements 4 extend radially more or less depending on the external diameter 81 to reach, so that the radius 82 is variable, while the footplates 5 keep the same geometry and size (prior to implantation). This avoids compression uncertainties on the footplates 5 if their radial length had to be changed depending on the external diameter 81. However, considering footplates 5 of variable radial length (with or without a radius 82 that may be constant) for reaching the desired external diameter 81 is also fully part of the invention.

The “best” vault height 89A inherent to the IOL 1 is determined at step (B) independently from the external diameter 81 of step (A). It is considered for this design step (B) only the dome K of the IOL 1. The inherent vault height 89A can be determined on basis of the ACD of the eye 9 preoperatively measured along the optical axis Z. The dome K is then customized so that the peripheral haptic part 3 extends radially outward to the cylinder of radius 82 (or also specifically so that the main proximal portion 34 extends radially outward to the cylinder of appropriate diameter 83) and posteriorly relative to the central optical part 2 of a depth (measured along the optical axis Z) corresponding to the inherent vault height 89A. Optionally, the dome K is based on the anterior surface radius of curvature of the crystalline lens.

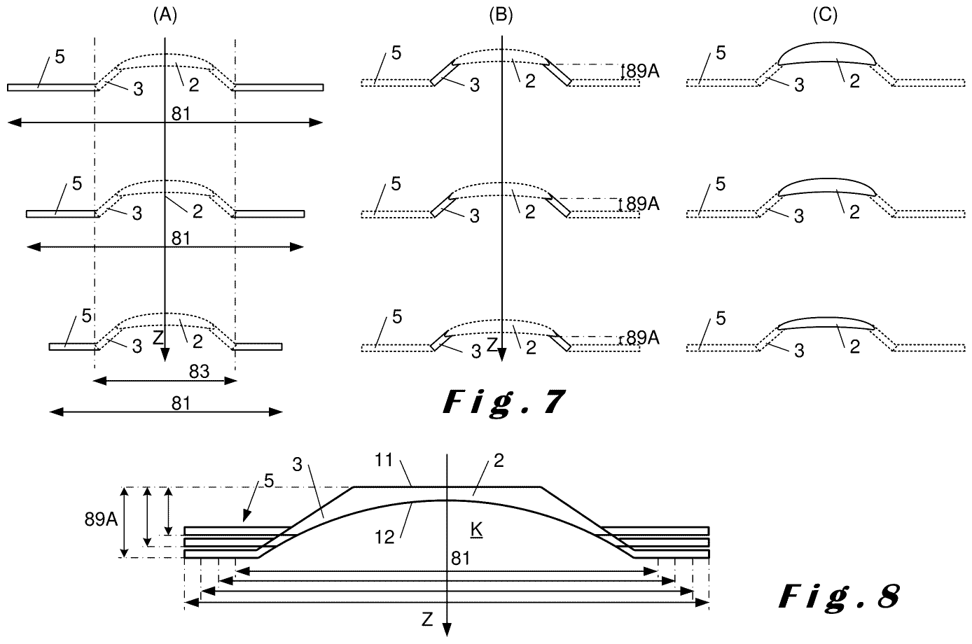

FIG. 8 further illustrates schematically possible independent (N=3)×(M=4) choices for the external diameter 81 and the inherent vault height 89A and their impact on the IOL 1 haptics structures.

Then, the appropriate central optical part 2 is chosen at step (C) based on the vision defect to be corrected for the eye 9, as known by the skilled person. As the anterior and posterior surfaces of the central optical part 2 define optical surfaces, those are designed depending on refractive efficiencies to be achieved. These surfaces may be customized to achieve a determined radius of curvature k for the dome K. Finally, after step (C), the IOL 1 can be manufacture according to the design resulting from steps (A), (B) and (C).

In brief, the invention relates to a phakic IOL 1 to be implanted into an eye's posterior chamber 96 which has an inherent vault height 89A independent from the IOL external diameter 81. The invention also relates to a manufacturing method of such an IOL 1. According to a preferred embodiment, the IOL 1 has a central optical part 2, a peripheral haptic part 3 having distal support elements 4 arranged for supporting the IOL 1 on the ciliary zonule 97 of the eye 9, footplates 5 mounted on the support elements 4, each having a distal border 53 arranged for stabilizing the IOL 1 into the ciliary body 98 of the eye 9, and more preferably manipulation pockets 6 on an IOL 1 anterior surface 11, each pocket 6 being associated with one of the footplates 5.

In the foregoing description, specific details are set forth to provide a thorough understanding of representative embodiments of the invention. It will be apparent to the skilled person, however, that the embodiments disclosed herein may be practiced without embodying all of the specific details. In some instances, well-known process steps have not been described in detail for not unnecessarily obscuring various aspects of the invention. It will also be obvious to the skilled person that the invention is not limited to specific embodiments illustrated and/or described above, but that its scope is more broadly defined by the claims that are hereinafter introduced.

1. A posterior chamber phakic intraocular lens (IOL) having a vault height inherent to the IOL which is independent from an external diameter of the IOL.

2. The posterior chamber phakic IOL according to claim 1, dimensioned to be implanted into a posterior chamber of an eye, so that:
the external diameter corresponds to a first function of a ciliary body-to-ciliary
body distance and/or associated distance of the eye measured perpendicularly to an optical axis of the IOL; and
the inherent vault height corresponds to a second function of an anterior chamber depth and/or associated depth of the eye measured along the optical axis.

3. The posterior chamber phakic IOL according to claim 1, comprising:
an anterior surface and a posterior surface;
a central optical part comprising a lens, and extending radially relative to an optical axis directed from the anterior surface to the posterior surface;
a peripheral haptic part circumferentially mounted on the central optical part and comprising distal support elements arranged for supporting the IOL on a ciliary zonule when the IOL is in an implantation position in an eye; and
footplates mounted on the peripheral haptic part and comprising a distal border arranged for stabilizing the IOL into a ciliary body when the IOL is in the implantation position in the eye,
wherein the peripheral haptic part extends radially outward and posteriorly relative to the central optical part of a depth depending on the inherent vault height, and wherein the footplates and/or the distal support elements extend radially of a length depending on the external diameter.

4. The posterior chamber phakic IOL according to claim 3, wherein the distal support elements are extending from a circle around the optical axis, the circle having a diameter independent of the external diameter of the IOL.

5. The posterior chamber phakic IOL according to claim 3, wherein the central optical part and the peripheral haptic part form a dome having a concave smooth posterior surface, the radius of curvature of which is independent of both the inherent vault height and the external diameter of the IOL.

6. The posterior chamber phakic IOL according to claim 3, wherein each of the footplates correspond to a flexible haptic elongated from a first extremity to a second extremity both directly mounted on one of the support elements.

7. The posterior chamber phakic IOL according to claim 3, comprising a manipulation pocket on the anterior surface, radially aligned with one of the footplates, and dimensioned for cooperating with a tip of a manipulation tool by a keyed engagement of the tip into the pocket, so that a moving of the footplate can be induced by a moving of the tool.

8. The posterior chamber phakic IOL according to claim 1, wherein the inherent vault height is chosen among N>1 possible values, and the external diameter of the IOL is chosen among M>1 other possible values.

9. A set of posterior chamber phakic intraocular lenses (IOLs), wherein a respective posterior chamber phakic IOL of the set has a vault height inherent to the IOL which is independent from an external diameter of the IOL, wherein the inherent vault height is chosen among N>1 possible values, the external diameter of the IOL is chosen among M>1 other possible values, and the set comprises N×M IOLs, all having distinct pair of inherent vault height and external diameter.

10. A method for choosing an intraocular lens (IOL) to be implanted into a posterior chamber of an eye among the set according to claim 9, the method comprising:
a first set of operations including:
measuring a ciliary body-to-ciliary body distance and/or associated distance of the eye perpendicularly to an optical axis of the IOL; and
selecting the external diameter of the IOL as the closest value to a first function of the measured distance among said M possible values;
a second set of operations including:
measuring an anterior chamber depth and/or associated depth of the eye along the optical axis; and
selecting the inherent vault height of the IOL as the closest value to a second function of the measured depth among said N possible values;
and a third set of operations including:
choosing the IOL among the set having the selected inherent vault height and external diameter.

11. A manufacturing method of a posterior chamber phakic intraocular lens (IOL), the method comprising:
(i) determining an external diameter of the IOL;
(ii) determining a vault height inherent to the IOL independently from the external diameter; and
(iii) manufacturing the IOL having the external diameter and the inherent vault height.

12. The manufacturing method according to claim 11, wherein the IOL is dimensioned to be implanted into a posterior chamber of an eye, wherein the manufacturing method comprises:
measuring a ciliary body-to-ciliary body distance and/or associated distance of the eye perpendicularly to an optical axis of the IOL before the step (i), and
measuring an anterior chamber depth and/or associated depth of the eye along the optical axis before the step (ii),
wherein the external diameter is determined at step (i) depending on the measured distance and the inherent vault height is determined at step (ii) depending on the measured depth.

13. The manufacturing method according to claim 11, further comprising, between the steps (ii) and (iii), designing optical surfaces of the IOL depending on refractive efficiencies to be achieved, wherein the IOL manufactured at step (iii) comprises these optical surfaces.

14. The manufacturing method according to claim 11, wherein the IOL comprises:
an anterior surface and a posterior surface;
a central optical part comprising a lens, and extending radially relative to an optical axis directed from the anterior surface to the posterior surface;
a peripheral haptic part circumferentially mounted on the central optical part and comprising distal support elements arranged for supporting the IOL on a ciliary zonule when the IOL is in an implantation position in an eye; and
footplates mounted the peripheral haptic part and comprising a distal border arranged for stabilizing the IOL into a ciliary body when the IOL is in the implantation position in the eye;
wherein the manufacturing method comprises:
between the steps (i) and (iii), designing the footplates and/or the distal support elements as extending radially of a length depending on the external diameter; and
between the steps (ii) and (iii), further designing the peripheral haptic part as extending radially outward and posteriorly relative to the central optical part of a depth depending on the inherent vault height.

15. The manufacturing method according to claim 11, wherein the IOL has a vault height inherent to the IOL which is independent from an external diameter of the IOL.
